(19) United States
(12) Patent Application Publication
(10) Pub. No.: US 2018/0049802 A1
(43) Pub. Date: Feb. 22, 2018

(54) CONTROLLED MULTI-NEEDLE POINT EXPANDING RADIOFREQUENCY ABLATION ELECTRODE NEEDLE

(71) Applicant: BEIJING INSTITUTE FOR CANCER RESEARCH, Beijing (CN)

(72) Inventors: Wei YANG, Beijing (CN); Minhua CHEN, Beijing (CN); Kun YAN, Beijing (CN); Song WANG, Beijing (CN)

(21) Appl. No.: 15/682,082

(22) Filed: Aug. 21, 2017

(30) Foreign Application Priority Data
Aug. 22, 2016 (CN) 2016107011792

(51) Int. Cl. A61B 18/14; A61B 17/00; A61B 18/08

(52) U.S. Cl. CPC A61B 18/1492; A61B 17/00234; A61B 18/20; A61B 2018/143; A61B 18/082

(57) ABSTRACT

A multi-needle point expanding radiofrequency ablation electrode needle, comprising a trocar, one end of which is provided with an opening for extending of needles, a handle portion fixedly arranged on the other end of the trocar and in which a plurality of slidable metallic sliding rods are provided, and a plurality of control sliding blocks that are slidably arranged on a peripheral wall of the handle portion. The present invention can control the number of the expanded needles, the extending direction of the needle and the extending length of the needle, such that an oriented expansion of electrode sub-needles, a complete ablation adapted to a tumor positioned below main vessels or in margin areas of organs and simplicity of operation are implemented.

This application claims priority to Chinese Patent Application No. 2016107011792, filed on Aug. 22, 2016, which is hereby incorporated by reference in its entirety.

### TECHNICAL FIELD

The present invention relates to an electrode needle and in particular to a multi-needle point expanding radiofrequency ablation electrode needle in the field of medical devices.

### BACKGROUND

Solid tumor is a common malignant disease harming human health, and treatment of tumors has become an important issue of improvement of survival time. In recent years, a local ablation technique for treatment of solid tumor represented by radiofrequency ablation (RFA) technique has developed rapidly. Tumor ablation indicates a direct application of chemotherapy or thermotherapy for some tumor (or several tumors) to eradicate or thoroughly destroy the tumor, such that the whole tumor including normal tissue having about 0.5-1.0 cm thickness on an outer circumferential thereof is completely solidified, necrotic and lose viability, a coagulative necrotic spheroid completely coating the whole tumor is established, and in which the target tumor is localized. The effect of removing tumor in the tumor ablation technique with tiny trauma is equivalent to non-tumor resection for the operation, thus it is a local treatment technique that is efficient, minimal invasive and useful for clinical work, wherein a successful ablation treatment includes factors in two aspects equilibrium with each other, in which one is to completely cover and ablate tumor tissues and a safety range around the tumor, and the other is to minimize damages to the neighboring normal tissues and other important structures.

RFA is one of the most widely used techniques in tumor ablation. A radiofrequency (RF) ablation instrument consists of an electrode needle, a measurement and control unit (e.g., a radiofrequency therapeutic generator) and a computer, etc., the electrode needle is connected with the measurement and control unit that automatically adjusts an output power of electrode needle radiofrequency ablation by monitoring changes in parameters of tumor tissues, such as impedance, temperature, etc, so that the tumor tissues quickly produce a large-scale coagulation necrosis. During treatment of tumor by the radiofrequency ablation instrument, the electrode needle is directly punctured tumor tissue under guidance of ultrasonic sound or CT. The electrode needle for radiofrequency ablation can cause a high temperature of the tissues to exceed 80° C. and cell death, so as to generate a coagulative necrosis area in tumor site. The electrode needle is a core component of the radiofrequency ablation instrument, as it directly affects size and shape of the coagulative necrosis area, and a desired shape of coagulative area should be spherical or elliposoidal with existing RFA electrodes.

The existing electrode needle for radiofrequency ablation includes a single-needle straight electrode (i.e., a single needle point straight electrode needle) and a multi-needle straight electrode (i.e., a multi-needle point straight electrode needle), as shown in FIG. 1 and FIG. 2, both the single-needle straight electrode and the multi-needle electrode are in a straight pathway, in operation, one or more electrode needles 1 are simultaneously inserted into lesions for ablation, but when a tumor 4 is located at a deep side of a blood vessel 3, as shown in FIG. 3, the straight needle electrode 1 is difficult to cross over a great vessel for ablating the tumor 4 at the deep side of the blood vessel 3, if it is desirable that a plurality of straight electrode needles 1 are distributed in parallel inside of the tumor 4, a needle placement is also difficult, an uneven distribution of spacing for the electrode needles 1 easily causes unevenness in thermal radiation distribution between the electrode needles 1, affects a necrosis range of tissues, as a result, the lesions ablation is incomplete, the tumor 4 is likely to remain, and the plurality of electrode needles 1 may have a plurality of points of puncture, which causes suffering to the patient, and it is also easy to puncture the blood vessel 3, and increase an incidence of hemorrhagic complications, moreover, it is difficult to select electrode needles for a fixed ablation range due to different sizes of multiple tumors.

The existing electrode needle for radiofrequency ablation further includes a multi-needle expandable electrode, as shown in FIGS. 4 to 6, the multi-needle expandable electrode includes a trocar 2 and 8-10 bendable arc-shaped electrode needles 1 located inside the trocar 2, the arc-shaped electrode needle 1 can extend from the end of the trocar 2, and is shaped as umbrella, anchor, Christmas tree or the like after expansion for treatment of spherical-like tumors, in operation, the trocar 2 is arranged in the center of the tumor 4, such that a plurality of electrode needles 1 are uniformly expanded to a perimeter area of the trocar 2, all the electrode needles 1 are uniformly distributed in 360 degrees by one operation of needle arrangement and can produce larger and reproducible necrotic lesions, and an ablation shape tends to be spherical, which overcomes a deficiency that a plurality of straight electrode needles need a plurality of points of puncture; however, when the tumor 4 is located at the deep side of the great vessel, the electrode needles 1 also cannot be completely disposed in the center position of the tumor 4 to completely cover and ablate the whole tumor 4 while avoiding the blood vessel 3, which results in an incomplete ablation of the tumor 4, and the plurality of electrode needles 1 expanded in spherical shape cause an easy damage to the blood vessels 3 and most of the normal tissues, that is, the existing multi-needle point expandable electrode needle fails to perform an oriented expansion so that the extending direction of the needle and the number of the expanded needles cannot be controlled.

Taking a hepatic tumor as an example, the existing radiofrequency ablation is a method for treatment of hepatic tumor at a lower risk, the mortality rate is only about 0.3% to 4.5%, the incidence of severe complications is about 2.2% to 8.9%, thus it has become an important means for treatment of small hepatocellular carcinoma or liver metastasis, however, as shown in FIGS. 3 and 6, since a liver 5 is of an irregular shape, there are more blood vessels 3 distributed inside the liver 5 and the periphery thereof is adjacent to organs such as gall bladder, stomach and intestine, and the like, the liver 5 is affected by structure and needle arrangement angle of the electrode 1 when the radiofrequency ablation technique is used for treatment, in this case, the tumor 4 positioned below the great vessels or in margin areas of the liver is less easily destroy thoroughly, even after the tumor 4 positioned below the great vessels or in margin areas of the liver undergoes a radiofrequency therapy, a local recurrence rate thereof is also higher than the recurrence rate of the tumor 4 in other positions, also, the complication mentioned above is mainly because of bleeding or damages to the surrounding organs, and reasons for occurrence are a mechanical damage or thermal damage of the electrode to the blood vessels or surrounding structures.

In views of the above-mentioned problems in the prior art, the inventor provides a controlled multi-needle point expanding radiofrequency ablation electrode needle in combination with the design in the related manufacture field and use experience over years, so as to overcome the drawbacks mentioned above.

### SUMMARY OF THE INVENTION

It is an object of the present invention to provide a controlled multi-needle point expanding radiofrequency ablation electrode needle, which can control the number of the expanded needles, the extending direction of the needle and the extending length of the needle, such that an oriented expansion of electrode sub-needles, a complete ablation adapted to tumor positioned below great vessels or in margin areas of organs and simplicity of operation are implemented.

The above-mentioned object of the present invention can be implemented using the technical solutions as follows:

The present invention provides a controlled multi-needle point expanding radiofrequency ablation electrode comprising: a trocar, one end of which is provided with an opening for extending of needles, wherein a plurality of electrode sub-needles that can extend from the opening for extending of needles, respectively, and expand and bend outwardly are threaded in the trocar, and the plurality of electrode sub-needles are circumferentially disposed in the trocar; a handle portion fixedly arranged on the other end of the trocar and in which a plurality of slidable metallic sliding rods are provided, one end of the metallic sliding rod is connected to the electrode sub-needle, and the other end of the metallic sliding rod is connected to a radiofrequency therapeutic generator; and a plurality of control sliding blocks that are slidably arranged on a peripheral wall of the handle portion, and the control sliding blocks are connected with the metallic sliding rods.

In a preferred embodiment, the trocar is provided with a plurality of radiofrequency electrode cannulas, which are circumferentially disposed in the trocar at equal intervals, and each of the electrode sub-needles is threaded in each of the radiofrequency electrode cannulas; in the state that the electrode sub-needles are positioned in the radiofrequency electrode cannulas, the electrode sub-needles are straight; and in the state that the electrode sub-needles extend from the opening for extending of needles of the trocar, the electrode sub-needle positioned outside the trocar is arc shaped.

In a preferred embodiment, a plane where the electrode sub-needle expanding from the opening for extending of needles expands and bends outwardly is co-planar with a sagittal plane of the control sliding block connected to the expanding electrode sub-needle. The control sliding block can control one or more expanding electrode sub-needles.

In a preferred embodiment, the trocar comprises an inner lumen, in which the plurality of radiofrequency electrode cannulas are circumferentially disposed at equal intervals and in which polyvinyl fluoride fillers are filled.

In a preferred embodiment, a plurality of slideways are uniformly circumferentially provided on the peripheral wall of the handle portion, the slideways are in parallel with a central axis of the trocar, and the control sliding blocks can be slidably disposed on the slideways.

In a preferred embodiment, the slideways are provided with a scale bar.

In a preferred embodiment, a plurality of sliding tracks are circumferentially provided in the handle portion, the metallic sliding rods are slidably provided on the sliding tracks.

In a preferred embodiment, the sliding tracks are provided with sliding beads, the metallic sliding rod is provided with a plurality of grooves at intervals in its sliding direction, and the sliding bead can be clipped within one of the plurality of grooves.

In a preferred embodiment, the grooves are five, a spacing between the grooves adjacent to each other is 1 cm, a diameter of an arc formed after the electrode sub-needles extend from the opening for extending of needles of the trocar and expand and bend outwardly is 1 cm to 5 cm.

In a preferred embodiment, needle tip of the electrode sub-needle is provided with a thermocouple temperature sensor.

Features and advantages of the controlled multi-needle point expanding radiofrequency ablation electrode needle of the present invention are:

1. In the present invention, a plurality of control sliding blocks are uniformly arranged in a circumferential direction of the handle portion, and an electrode sub-needle is individually controlled by each control sliding block for implementation of independent extension or retraction of each electrode sub-needle, so as to control the number of the extended needles, the extending direction of the needle and the extending length of the needle of the plurality of electrode sub-needles, overcome the defect that a traditional straight needle electrode and a multi-needle electrode cannot change the number and the extending direction of the needle, realize a controlled and oriented expansion of the electrode sub-needles, improve an accuracy of treatment of targets, realize an accurate radiofrequency ablation, prevent unnecessary damages, and have advantages of strong targeting ability, good local effect, low complications, flexible operation and ease of adjustment;

2. In the present invention, the plane where the electrode sub-needles expand and bend outwardly is co-planar with the sagittal plane of the control sliding blocks to facilitate control and indication of the expansion and bending direction of the electrode sub-needles, and a scale bar on the slideways and scales on the trocar are provided, so as to facilitate observation of an insertion depth of the trocar in operation, and the extending length of the electrode sub-needles or the expanding and bending diameter after expanding, meanwhile the slide beads on the sliding tracks are engaged with the plurality of grooves on the metallic sliding rods, so as to facilitate controlling and sensing of the extending length of the electrode sub-needles; and

3. In the present invention, the trocar can be disposed at the margin of the tumor at once, and one or more electrode sub-needles may be expanded and bended toward the direction of the tumor, such that the tumor is more accurately covered in the radiofrequency ablation scope, and according to the size and morphology of the tumor, and the distribution of peripheral blood vessels and organs, the electrode sub-needles in an appropriate direction can be selected for expansion and bending of a suitable length, which effectively avoids the peripheral blood vessels and organs, decreases damages to normal organs and blood vessels, reduces the incidence rate of bleeding and other complications, particularly in the case that the tumor is located at a deep side of great vessels, the tumor is adjacent to the organs or the tumor in the organs is a multiple tumor and is different in size, a variety of tumors in different sizes can be treated by controlling the extending length of the needle and the extending direction of the needle of the electrode sub-needle by the control sliding blocks to decrease residue of tumor, reduce the patient pain and thus it is applied widely.

### BRIEF DESCRIPTION OF THE DRAWINGS

In order to more clearly illustrate the technical solution of the embodiments of the present invention, the following will briefly introduce the accompanying drawings to be used in the description of the embodiments. Obviously, the accompanying drawings in the following description are merely some embodiments of the present invention, it will be apparent to those skilled in the art without the exercise of inventive faculty to further obtain other accompanying drawings based on those ones.

FIG. 1 is a structural diagram illustrating the existing single needle straight electrode;

FIG. 2 is a structural diagram illustrating the existing multi-needle straight electrode;

FIG. 3 is a diagram illustrating an application of the existing single needle straight electrode to hepatic tumor treatment which is located on the edge of the liver or in deep site of large vessels;

FIG. 4 is a structural diagram illustrating an embodiment of the existing multi-needle point expandable electrode needle;

FIG. 5 is a structural diagram illustrating another embodiment of the existing multi-needle point expandable electrode needle;

FIG. 6 is a diagram illustrating an application of the existing multi-needle point expandable electrode needle in tumor therapy;

FIG. 7a is a structural diagram illustrating the controlled multi-needle point expandable radiofrequency ablation electrode needle of the present invention;

FIG. 7b is another structural diagram illustrating the controlled multi-needle point expandable radiofrequency ablation electrode needle of the present invention, at different view;

FIG. 8a is a structural diagram illustrating the trocar of the controlled multi-needle point expandable radiofrequency ablation electrode needle of the present invention;

FIG. 8b is another structural diagram illustrating the trocar of the controlled multi-needle point expandable radiofrequency ablation electrode needle of the present invention, at different view;

FIG. 9a is a local structural diagram illustrating the trocar of the controlled multi-needle point expandable radiofrequency ablation electrode needle of the present invention;

FIG. 9b is another local structural diagram illustrating the trocar of the multi-needle point expandable radiofrequency ablation electrode needle of the present invention, at different view;

FIG. 10 is a schematic diagram illustrating the cross section of the controlled multi-needle point expandable radiofrequency ablation electrode needle of the present invention with none of its electrode sub-needles expanded;

FIG. 11 is a schematic diagram illustrating the cross section of the controlled multi-needle point expandable radiofrequency ablation electrode needle of the present invention with all of its electrode sub-needles expanded;

FIG. 12 is a structural diagram illustrating the handle portion of the controlled multi-needle point expandable radiofrequency ablation electrode needle of the present invention;

FIG. 13 is a section view through section line A-A of FIG. 12;

FIG. 14a is a structural diagram illustrating connection of the electrode sub-needles, metallic sliding rods, and control sliding blocks of the present invention;

FIG. 14b is another structural diagram illustrating connection of the electrode sub-needles, metallic sliding rods, and control sliding blocks of the present invention; and

FIG. 15 is a structural diagram illustrating the control sliding blocks and the slideways of the present invention.

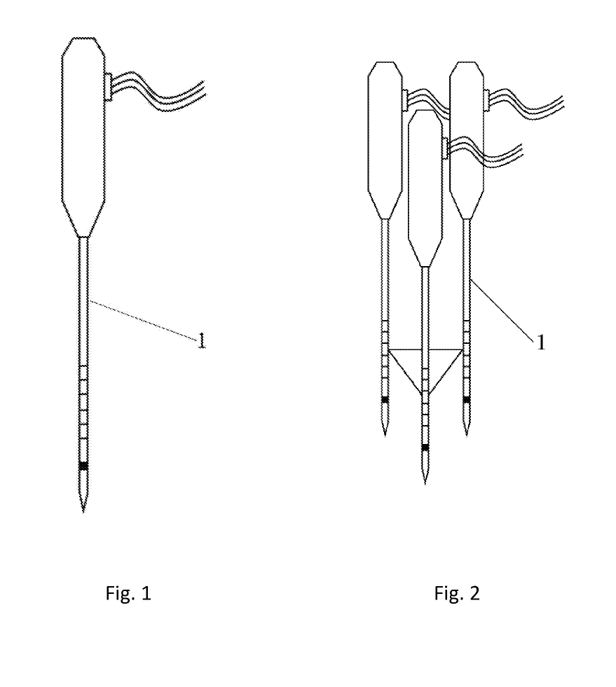

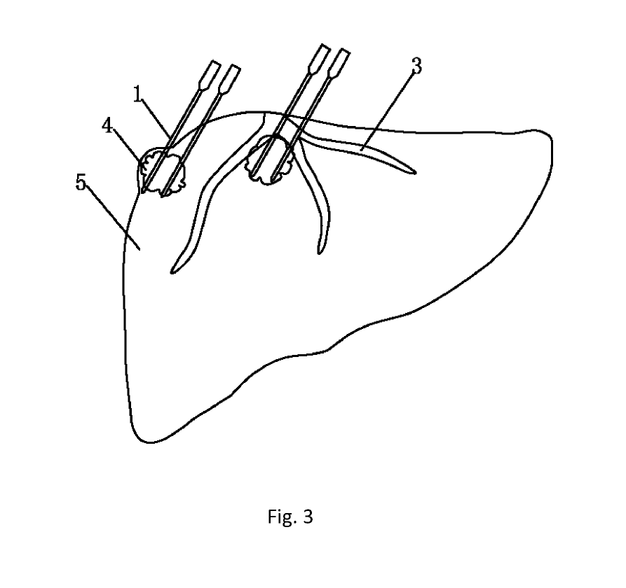

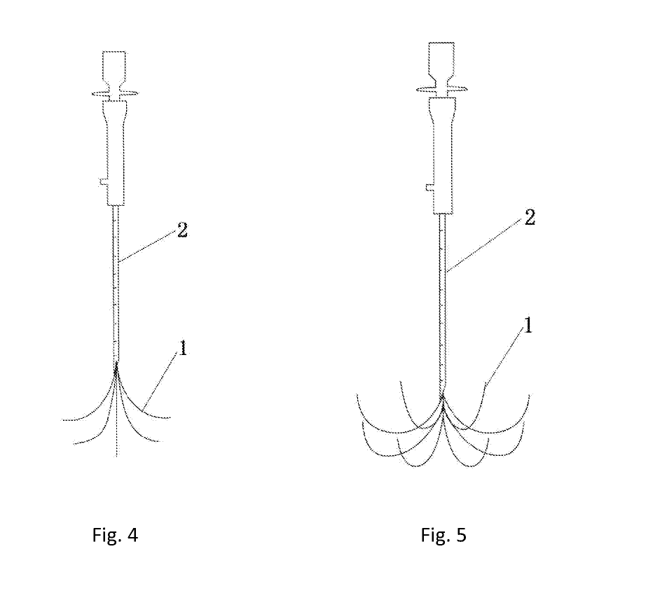

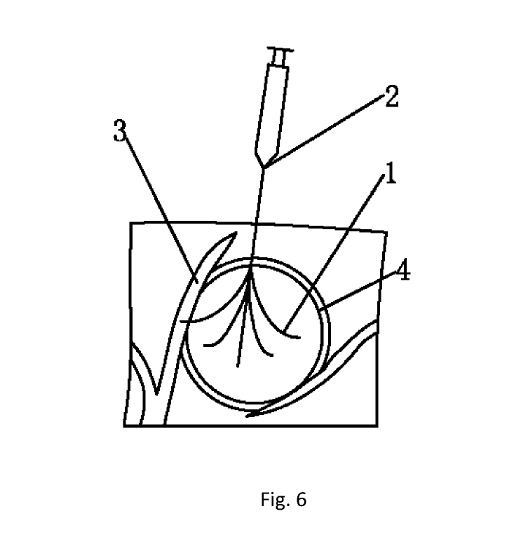

### DESCRIPTION OF REFERENCE NUMERALS

### Prior Art

1. radiofrequency ablation electrode needle; 2. trocar; 3. blood vessel; 4. tumor; and 5. liver.

### The Present Invention

10. electrode sub-needle; 11. needle tip; 12. trocar; 13. end for extending of needles; 14. fixed end; 15. base; 16. radiofrequency electrode cannula; 17. filler; 18. handle portion; 19. slideway; 20. control sliding block; 21. connection post; 22. metallic sliding rod; 23. groove; 24. sliding track; 25. sliding bead; 26. filler; 27. power connector; and 28. connection cable to generator.

### Detailed Description of the Invention

The technical solutions in the embodiment of the present invention are clearly and completely described in conjunction with the accompanying drawings in the embodiment of the present invention, and it is apparent that these depicted embodiments are only some but not all of the embodiments of the present invention. All the other embodiments obtained by those skilled in the art without the exercise of inventive faculty fall under the protective scope of the present invention, based on the embodiments of the present invention.

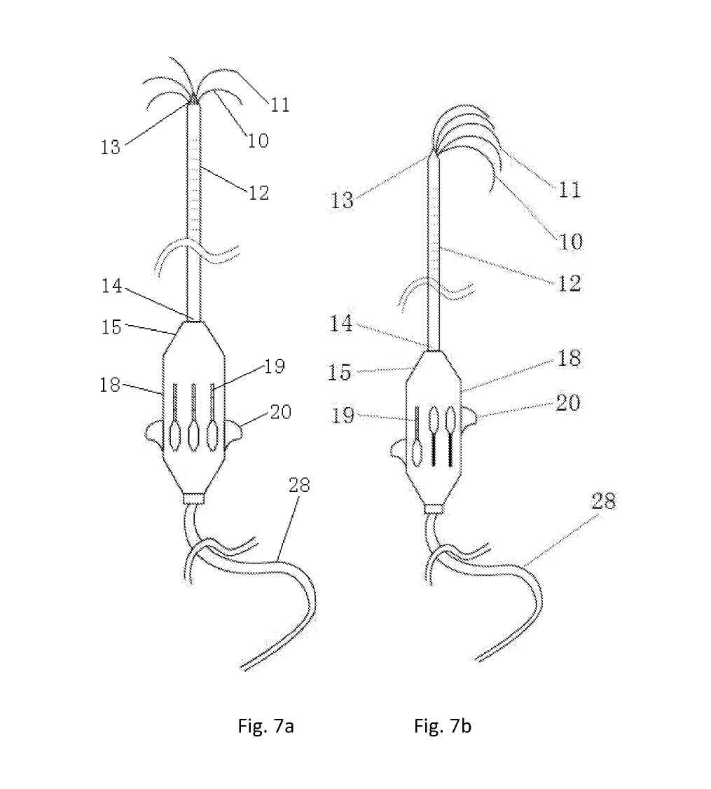

Unless the direction is individually defined and pointed out, as for the directions “left”, “right”, “up”, “down” referred to herein, reference is made to the directions “left”, “right”, “up”, “down” shown in FIG. 7a in the present invention to be described herein.

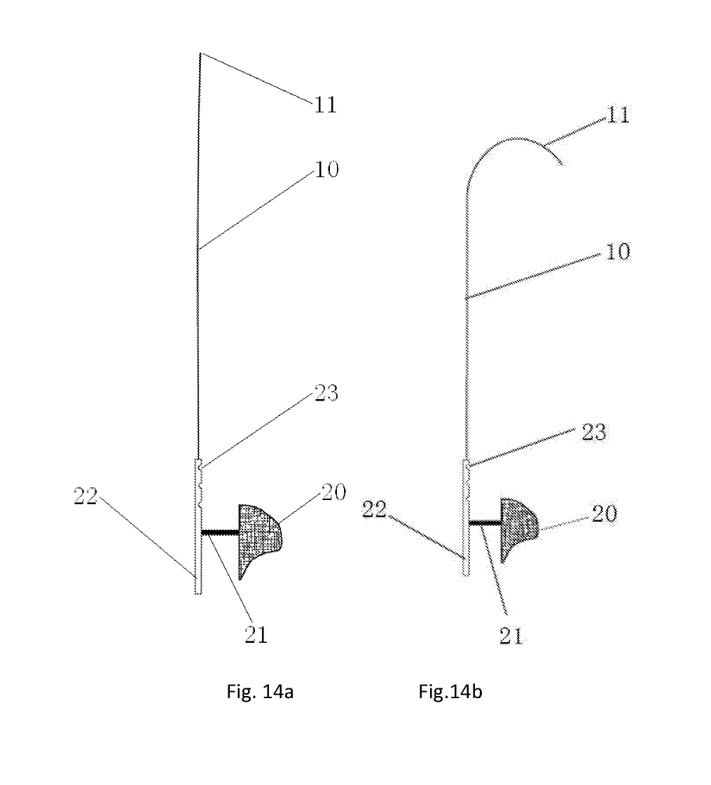

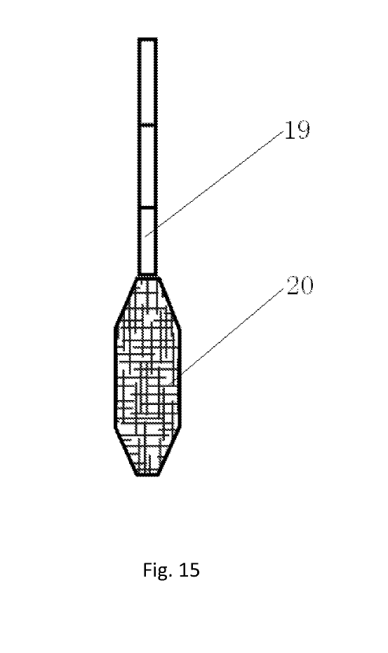

As shown in FIG. 7a to FIG. 15, the present invention provides a multi-needle point expanding radiofrequency ablation electrode needle comprising: a trocar 12, one end of which is provided with an opening for extending of needles, wherein a plurality of electrode sub-needles 10 that can extend from the opening for extending of needles, respectively, and expand and bend outwardly are threaded in the trocar 12, and the plurality of electrode sub-needles 10 are circumferentially disposed in the trocar 12; a handle portion 18 fixedly arranged on the other end of the trocar 12 and in which a plurality of slidable metallic sliding rods 22 are provided, one end of the metallic sliding rod 22 is connected to the electrode sub-needle 10, and the other end of the metallic sliding rod 22 is connected to a radiofrequency therapeutic generator; and a plurality of control sliding blocks 20 that are slidably arranged on a peripheral wall of the handle portion 18, and the control sliding blocks 20 are connected with the metallic sliding rods 22.

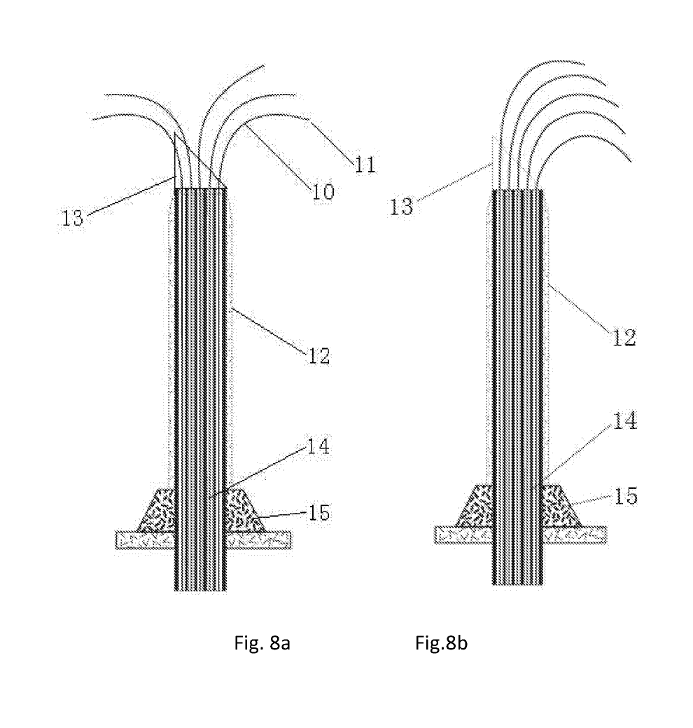

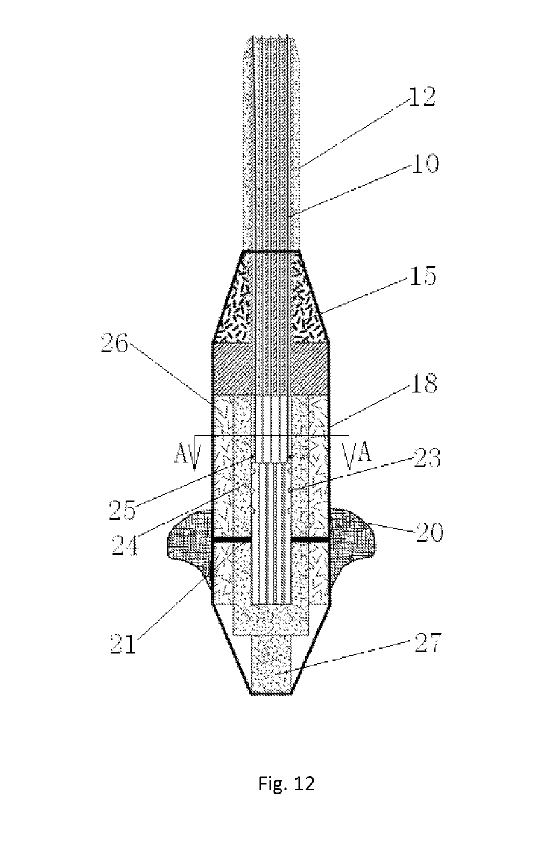

In particular, as shown in FIGS. 8a and 12, the trocar 12 is in an elongated cylindrical shape, preferably, an external diameter of the trocar 12 is 14 G-18 G (wherein G is an abbreviation for Gauge), one end of the trocar 12 (i.e., an upper end in FIG. 7a and FIG. 7b) is an end for extending of needles 13 that is provided with the opening for extending of needles, in order to facilitate the extension and the retraction of the plurality of electrode sub-needles 10, the other end of the trocar 12 is a fixed end 14 on which a base 15 is arranged, and the base 15 is connected to the handle portion 18 so that the trocar 12 is secured onto the handle portion 18 to prevent the trocar 12 from coming out, an outer peripheral wall of the trocar 12 is provided with scales to indicate an insertion depth of the trocar 12, the outer peripheral wall of the trocar 12 is coated with an ultra-thin compact insulation and anti-blocking nanofiber coating, particularly the end for extending of needles 13 of the trocar 12, so that the trocar 12 and the end for extending of needles 13 thereof can be clearly shown under ultrasound. Naturally, the end for extending of needles 13 can also be directly made of a material that can be shown under ultrasound, which is not limiting herein.

Further, as shown in FIGS. 8a to 11, the trocar 12 is provided with a plurality of radiofrequency electrode cannulas 16, which are circumferentially disposed in the trocar 12 at equal intervals, and each of the electrode sub-needles 10 is threaded in each of the radiofrequency electrode cannulas 16; the electrode sub-needles 10 have an ultra strong memory function and a flexibility; in the state that the electrode sub-needles 10 are positioned in the radiofrequency electrode cannulas 16, the electrode sub-needles 10 are straight; and in the state that the electrode sub-needles 10 extend from the opening for extending of needles of the trocar 12, the electrode sub-needle 10 positioned outside the trocar 12 is arc shaped. Particularly, the material of the radiofrequency electrode cannula 16 is polytetrafluorethylene (PTFE) that has a good slidability, a surface of the electrode sub-needles 10 has a stronger sound reflection coating, such that the electrode sub-needles 10 can be clearly shown under ultrasound, or the electrode sub-needles 10 are directly made of the material that can be shown under ultrasound, for example, the electrode sub-needles 10 are made of a titanium alloy material, the electrode sub-needles 10 using such material can be in an arcuately curved state when the electrode sub-needles 10 extend from the opening for extending of needles of the trocar 12, the arc of each electrode sub-needle 10 can be sector, semicircle, nearly close to circular or the like after extending from the opening for extending of needles, and expanding and bending outwardly, and after all of the electrode sub-needles 10 extend from the opening for extending of needles, and expand and bend outwardly, the portion formed when all of the electrode sub-needles 10 expand and bend is in an umbrella shape, an anchor shape, a hemispherical or nearly close to a spherical shape, preferably, the electrode sub-needles 10 are six, eight or ten, such that a distance between the electrode sub-needles 10 adjacent to each other, or the space surrounded by a plurality of the electrode sub-needles 10 can exactly satisfy requirements of radiofrequency treatment (i.e., the tumor can be completely covered) after the electrode sub-needles 10 extend, and expand and bend outwardly, and it is apparent that the electrode sub-needles 10 can also be arranged in other suitable numbers based on treatment demands, which is not limited herein.

Further, one end that the electrode sub-needle 10 can extend from the opening for extending of the needles is its needle tip 11 that is provided with a high-sensitive thermocouple temperature sensor, which can feed back a signal to a control computer for real-time display of temperature of the needle tip 11 of each electrode sub-needle 10 during radiofrequency; the other end of the electrode sub-needle 10 opposite to the needle tip 11 is connected to the metallic sliding rod 22 such that the extension and the retraction of the electrode sub-needles 10 are controlled by the metallic sliding rods 22, and so as to transmit the radio frequency of radiofrequency therapeutic apparatus to the electrode sub-needles 10, preferably, the electrode sub-needles 10 and the metallic sliding rods 22 can be connected to each other by welding, and they can be surely connected to each other by other means, which is not limited herein.

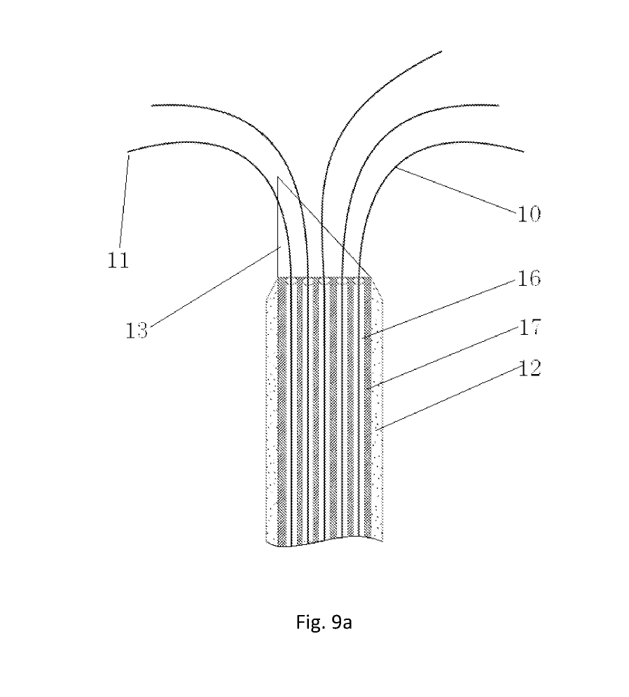

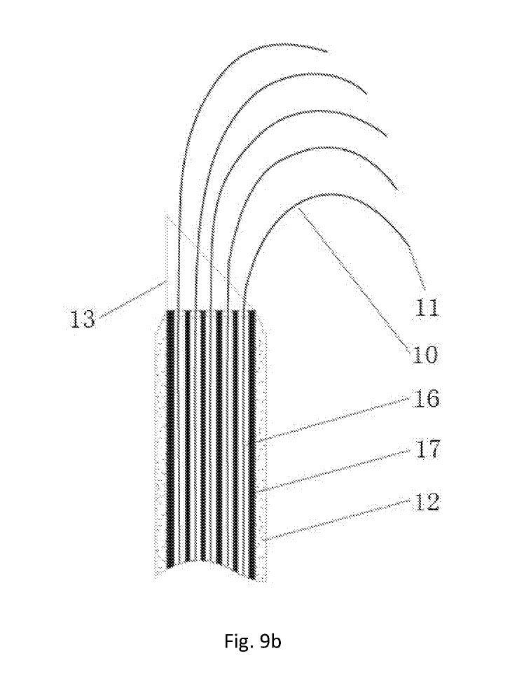

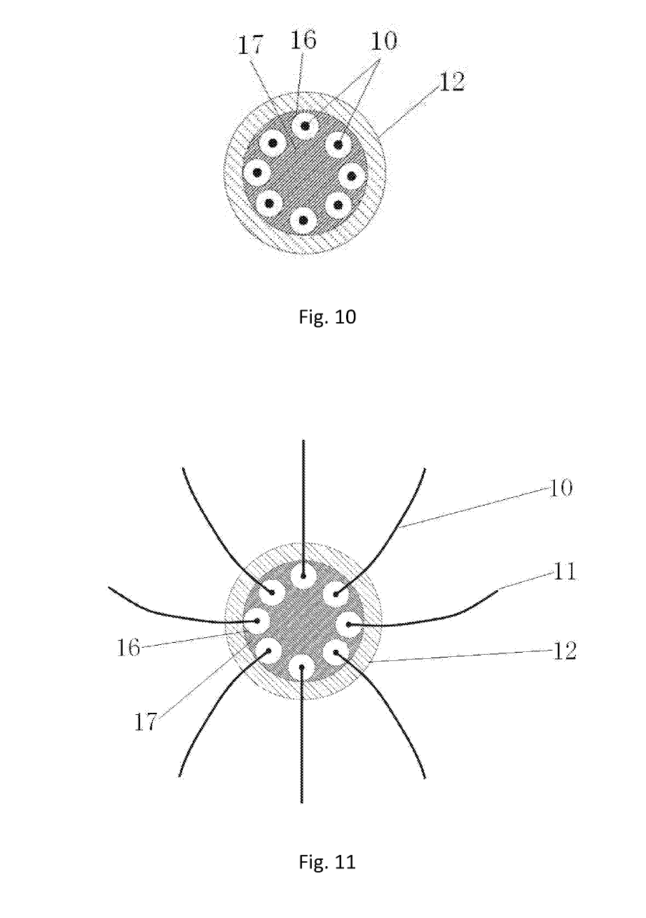

Further, in one embodiment, the trocar 12 is a solid cylinder having a plurality of axial through holes that are uniformly arranged in the circumferential direction of the trocar 12, and a plurality of radiofrequency electrode cannulas 16 are located in the plurality of through holes; as shown in FIGS. 9a and 10, in another embodiment, the trocar 12 has an inner lumen, in which the plurality of radiofrequency electrode cannulas 16 are circumferentially disposed at equal intervals and in which polyvinyl fluoride fillers 17 are filled, and the position of a plurality of radiofrequency electrode cannulas 16 remains fixed without change by the polyvinyl fluoride fillers 17, so as to ensure a smooth and easy extending of electrode sub-needles 10, and it is sure to also arrange other filling materials, as long as the fixing of the plurality of radiofrequency electrode cannulas 16 can be guaranteed, which is not limited herein.

Further, as shown in FIGS. 7a and 7b, a plane where the electrode sub-needle 10 expanding from the opening for extending of needles expands and bends outwardly is co-planar with a sagittal plane of the control sliding block 20 connected to the extending electrode sub-needle 10, particularly, as shown in FIG. 11, when the plurality of electrode sub-needles 10 uniformly arranged in the circumferential direction in the trocar 12 all extend from the opening for extending of needles, the plurality of electrode sub-needles 10 expand and bend outwardly along the respective curved path, respectively, the plane where each electrode sub-needle 10 expands and bends is perpendicular to a radial cross-section of the trocar 12, and a central axis of the trocar 12 is located on the plane where each electrode sub-needle 10 expands and bends, the sagittal plane of the control sliding block 20 is a plane which divides the control sliding block 20 into two symmetrical portions in the direction of the central axis of the trocar 12; it can implement control and indication of the direction in which the electrode sub-needles 10 expand and bend using the control sliding blocks 20 by allowing the two planes to be coplanar, which is simple to handle. One control sliding block can decide the direction and length of one sub-needle or multiple sub-needles depend on the feasibility of electrode production.

Further, as shown in FIGS. 7a and 12, a plurality of slideways 19 are uniformly circumferentially provided on the peripheral wall of the handle portion 18, the slideways 19 are in parallel with the central axis of the trocar 12, and the control sliding blocks 20 can be slidably disposed on the slideways 19. Particularly, the slideways 19 are grooves hollowly disposed on the peripheral wall of the handle portion 18, the control sliding blocks 20 on the slideways 19 are connected to the metallic sliding rods 22 by connection posts 21, wherein the distribution of the slideways 19 and the control sliding blocks 20 in the circumferential direction each corresponds to that of the electrode sub-needles 10 in the circumferential direction, and the number of the slideways 19 and the control sliding blocks 20 is identical to that of the electrode sub-needles 10, such that when each control sliding block 20 slides along the respective slideway 19, a synchronous sliding of the corresponding metallic sliding rods 22 is driven by the connection posts 21 to control the extension or retraction of the corresponding electrode sub-needles 10; preferably, the slideway 19 is provided with a scale bar to show whether the control sliding block 20 slide and a length of sliding and hence whether the corresponding electrode sub-needle 10 extends and an extending length of the needle can be appreciated, wherein a length of the scale bar is set based on practical requirements, which is not limited herein; in one embodiment, the scale bar can be uniformly divided into five scales, an interval between the scales adjacent to each other is 1 cm, once the control sliding block 20 slides by one scale towards the direction close to the opening for extending of the needles of the trocar 12 along the slideway 19, the metallic sliding rod 22 also slides by the same length, and drives the electrode sub-needle 10 to extend the same length, that is, the length by which the control sliding block 20 slides is namely the length that the electrode sub-needle 10 extends; in another embodiment, the scale bar is divided into five scales, each of which corresponds to a diameter in a grade of which the electrode sub-needle 10 expands and bends after extending, that is, when the control sliding block 20 slides by per scale of unit length, the diameter of which the electrode sub-needles 10 expands and bends is increased or decreased by one unit length, and it is apparent that the scales on the scale bar can also be unevenly arranged, as long as they can correspond to the range in which the electrode sub-needle 10 extends in a tumor.

Further, as shown in FIG. 12, a plurality of sliding tracks 24 are circumferentially provided in the handle portion 18, the metallic sliding rods 22 are slidably provided on the sliding tracks 24, wherein an extending direction of the sliding track 24 is parallel to the central axis of the trocar 12, and the number of the sliding tracks 24 and that of the metallic sliding rods 22 are identical and share an one-to-one correspondence, such that when each of the metallic sliding rods 22 slides in the respective sliding track 24 to drive an extension or retraction of the electrode sub-needle 10 connected thereto, respectively; in one embodiment, the handle portion 18 is substantially in the shape of a cylinder and has an inner lumen, a plurality of sliding tracks 24 are circumferentially disposed in the inner lumen of the handle portion 18 at equal intervals and in which polyvinyl fluoride (PVC) fillers 26 are filled, which assures a stable interior structure and the sliding tracks 24 fixed, and assures a smooth sliding of the metallic sliding rods 22, and it is apparent that the filling materials can also be other materials; in another embodiment, the handle portion 18 is a solid cylinder having a plurality of axial through holes, and the plurality of sliding tracks 24 are provided in the plurality of through holes of the handle portion 18; it is more preferable that the sliding tracks 24 are made of metallic materials, one end where the sliding track 24 remotes from the needle tip 11 is connected to a power connector 27, and the power connector 27 is connected to the radiofrequency therapeutic generator via a connection cable 28, which achieves a transmission of radio-frequency energy to the electrode sub-needles 10.

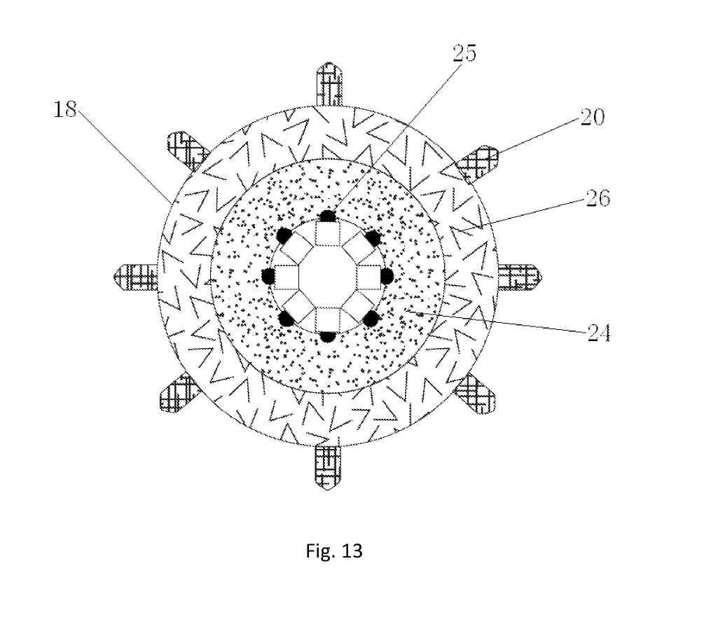

Further, as shown in FIGS. 12 and 13, the sliding tracks 24 are provided with sliding beads 25, the metallic sliding rod 22 is provided with a plurality of grooves 23 at intervals in its sliding direction, and the sliding bead 25 can be clipped within one of the plurality of grooves 23, wherein it is preferred that the depth of the grooves 23 preferably allows the sliding beads 25 to be clipped but not resists sliding of the metallic sliding rods 22, when the electrode sub-needles 10 do not extend, the sliding beads 25 on the sliding tracks 24 are located in front of the sliding path of the metallic sliding rods 22 (i.e., “on the top” in FIG. 12), during the extension of the electrode sub-needles 10 due to the sliding of the metallic sliding rods 22, the plurality of grooves 23 pass by the sliding beads 25 in turn, and are separated from the beads 25 following engagement, such a procedure provides an abortive feedback to an operator who operates the control sliding blocks 20, and thus the operator can sense the extending length of the needle of electrode sub-needles 10, and meanwhile it can further function to assist in positioning; and it is more preferable that the sliding beads 25 are pure copper ones and it is apparent that the beads 25 can be made of other materials, which is not limited herein.

In one embodiment, a spacing between the grooves 23 adjacent to each other is equal; more preferably, the grooves 23 are five, the spacing between the grooves 23 adjacent to each other is 1 cm, such that the control sliding blocks 20 slide by 1 cm to drive sliding of the metallic sliding rods 22 by 1 cm as well, and hence to drive an extension or retraction of the electrode sub-needles 10 by 1 cm, and meanwhile, a diameter of an arc formed after the electrode sub-needles 10 extend from the opening for extending of needles of the trocar 12 and expand and bend outwardly is 1 cm to 5 cm, a change in diameter of the electrode sub-needles 10 corresponds to the distance by which the control sliding blocks 20 slide; in another embodiment, the spacing between the grooves 23 adjacent to each other could not be equal but merely correspond to the range in which the electrode sub-needles 10 extend in a tumor, e.g., the spacing between the grooves 23 adjacent to each other corresponds to the diameter of which the electrode sub-needles 10 expand and bend, meanwhile, the number of the grooves 23 can also be arranged based on practical requirements, which is not limited herein.

Prior to an application of the controlled multi-needle point expanding radiofrequency ablation electrode needle of the present invention for treatment, first of all, examining and assessing the number, size, shape, position of a tumor and the relation with peripheral great vessels and nearby important organs by ultrasound, determining an insertion point (i.e., target) of the trocar 12, next designing an advancement direction and an advancement angle of the electrode sub-needles 10 in conjunction with guidelines, so that the electrode sub-needles 10 can cover the entire tumor and stay away from the great vessels and nearby important organs; during treatment, under ultrasound guidance, inserting the electrode sub-needles 10 into the target along the designed route, determining an extending angle of the electrode sub-needles 10 based on the positional relation between the tumor and the trocar 12, and determining the number and length of the electrode sub-needles 10 desired to be expandable according to the size of tumor and the extending angle of the needle of the electrode sub-needles 10, the following two situations need to be specifically described:

when a tumor is located at a deep side of the great vessels or at the margin of nearby organs, the target is set as a margin where a end of the tumor stays away from the great vessels and organs, first of all, inserting the trocar 12, such that the end for extending of needles 13 of the trocar 12 is placed in the set target position; next, performing an operation of extending of the needle for the electrode sub-needles 10 that can expand and bend towards the tumor, operating the control sliding blocks 20 to move towards the end for extending of needles 13 on its slideways 19, such that the metallic sliding rods 22 connected thereto slide together with the control sliding blocks 20 to drive an extension and expansion and bend of the connected electrode sub-needles 10 and the control sliding blocks 20 allow for movement per scale, i.e., it represents that the electrode sub-needles 10 extend by one scale or the diameter of the electrode sub-needles 10 is increased by one scale, and it also represents that an ablation range is constantly expanding until the electrode sub-needles 10 expand from one end of the tumor to the other end, and moving the control sliding blocks 20 is stopped, during which, size and morphology of the tumor and distribution of the nearby vessels and organs need to be considered and the extending direction of the electrode sub-needles 10 is controlled, such that it is biased towards the tumor exactly from one end of the tumor and avoids damaging the nearby blood vessels and organs, and it is preferred for the extending length that the electrode sub-needles 10 slightly cover the tumor (e.g., the tumor is 3 cm, the electrode sub-needles 10 may be expanded to 4 cm), accordingly, a needle arrangement of a plurality of electrode sub-needles 10 is completed in turn, upon completion of the needle arrangement, the radiofrequency therapeutic generator is turned on to complete the radiofrequency ablation by the connection cable 28 providing a radio-frequency transmission to the electrode sub-needles 10; and finally, the radiofrequency therapeutic generator is stopped, and the control sliding blocks 20 are operated to withdraw the electrode sub-needles 10 and then the trocar 12 is pulled out.

In the case that a tumor stays away from a deep side of the great vessels or at the margin of other organs, the target is set as a center of the tumor, first of all, inserting the trocar 12, such that the end for extending of needles 13 of the trocar 12 is placed in the target position, and an insertion depth can be observed with the scales on the trocar 12 in the insertion procedure, and then all the control sliding blocks 20 are operated to move on the slideways 19, such that the control sliding blocks 20 drive the metallic sliding rods 22 to move on the sliding tracks 24, and hence drive each electrode sub-needle 10 to extend from the opening for extending of needles, and expand and bend outwardly, a curved portion of the expanding electrode sub-needles 10 is substantially spherical to cover the entire scope of tumor and implement a complete ablation.

The present invention is applicable to treatment of tumors in any position in solid organs in a thermal ablation for treatment of organs, mainly in liver, and of course in spleen, kidney, mammary gland and thyroid, in particular, it is more specific to the tumor located in special positions of organs, e.g., the tumor located at a deep side of the great vessels and at the margin of the organs, multiple tumor in different sizes and the like where the existing electrode needles hardly manage. Of course, the structure of the present invention can also be used for microwave ablation, laser ablation and cryo-ablation techniques, the principle thereof is similar to radiofrequency ablation, so the detailed description thereof will be omitted.

Features and advantages of the multi-needle point expanding radiofrequency ablation electrode needle of the present invention are:

1. In the present invention, a plurality of control sliding blocks 20 are uniformly arranged in a circumferential direction of the handle portion 18. An independent or dependent extension or retraction of each electrode sub-needle 10 is implemented by each of the control sliding blocks 20 individually controlling one or more electrode sub-needle 10, so as to control the number of the extended needles, the extending direction of the needle and the extending length of the needle of the plurality of electrode sub-needles 10, overcome the defect that a traditional straight needle and a multi-needles expanding electrode cannot change the number of the extended needles and the extending direction of the needles, realize a controllable and oriented expansion of the electrode sub-needles 10, improve an accuracy of treatment of targets, realize an accurate radiofrequency ablation, prevent unnecessary damages in other direction tissues, and have advantages of strong targeting ability, good local effect, low complications, flexible operation and ease of adjustment;

2. In the present invention, the plane where the electrode sub-needles 10 expand and bend outwardly is co-planar with the sagittal plane of the control sliding blocks 20 to facilitate control and indication of the expansion and bending direction of the electrode sub-needles 10, and a scale bar on the slideways 19 and scales on the trocar 12 are provided, so as to facilitate observation of an insertion depth of the trocar 12 in operation, and the extending length of the electrode sub-needles 10 or the expanding and bending diameter after expanding, meanwhile the slide beads 25 on the sliding tracks 24 are engaged with the plurality of grooves 23 on the metallic sliding rods 22, so as to facilitate controlling and sensing of the extending length of the electrode sub-needles 10; and

3. In the present invention, the trocar 12 can be disposed at the margin of the tumor at once, and one or more electrode sub-needles 10 may be expanded and bended toward the direction of the tumor, such that the tumor is more accurately covered in the radiofrequency ablation scope, and according to the size and morphology of the tumor, and the distribution of peripheral blood vessels and organs, the electrode sub-needles 10 in an appropriate direction can be selected for expansion and bending of a suitable length, which effectively avoids puncturing the peripheral blood vessels and organs, decreases damages to normal organs and blood vessels, reduces the incidence rate of bleeding and other complications, particularly in the case that the tumor is located at a deep side of great vessels, the tumor is adjacent to the important structures or the tumor in the organs are multiple tumors and is different in size, a variety of tumors in different sizes can be treated by controlling the extending length of the needle and the extending direction of the needle of the electrode sub-needles 10 by the control sliding blocks 20 to decrease residual of tumor, reduce the patient pain and thus it is applied widely.

Although a specific embodiment of the invention has been described herein in detail, this has been done solely for the purpose of illustrating the invention in several of its aspects, and it is to be understood that the foregoing description does not limit the scope of the invention. It is contemplated that various substitutions, alterations and/or modifications to the embodiment of the invention disclosed herein, including but not limited to those implementation options specifically noted herein, may be made to the invention without departing from the spirit and scope of the invention as defined in the appended claims.

1. A controlled multi-needle point expanding radiofrequency ablation electrode needle, characterized by comprising:
a trocar, one end of which is provided with an opening for extending of needles, wherein a plurality of electrode sub-needles that can extend from the opening for extending of needles, respectively, and expand and bend outwardly are threaded in the trocar, and the plurality of electrode sub-needles are circumferentially disposed in the trocar;
a handle portion fixedly arranged on the other end of the trocar and in which a plurality of slidable metallic sliding rods are provided, one end of the metallic sliding rod is connected to the electrode sub-needle, and the other end of the metallic sliding rod is connected to a radiofrequency therapeutic generator; and
a plurality of control sliding blocks that are slidably arranged on a peripheral wall of the handle portion, and the control sliding blocks are connected with the metallic sliding rods.

2. The controlled multi-needle point expanding radiofrequency ablation electrode needle according to claim 1, characterized in that, the trocar is provided with a plurality of radiofrequency electrode cannulas, which are circumferentially disposed in the trocar at equal intervals, and each of the electrode sub-needles is threaded in each of the radiofrequency electrode cannulas; in the state that the electrode sub-needles are positioned in the radiofrequency electrode cannulas, the electrode sub-needles are straight; and in the state that the electrode sub-needles extend from the opening for extending of needles of the trocar, the electrode sub-needle positioned outside the trocar is arc shaped.

3. The controlled multi-needle point expanding radiofrequency ablation electrode needle according to claim 1, characterized in that, a plane where the electrode sub-needle expanding from the opening for extending of needles expands and bends outwardly is co-planar with a sagittal plane of the control sliding block connected to the expanding electrode sub-needle.

4. The controlled multi-needle point expanding radiofrequency ablation electrode needle according to claim 2, characterized in that, the trocar comprises an inner lumen, in which the plurality of radiofrequency electrode cannulas are circumferentially disposed at equal intervals and in which polyvinyl fluoride fillers are filled.

5. The controlled multi-needle point expanding radiofrequency ablation electrode needle according to claim 1, characterized in that, a plurality of slideways are uniformly circumferentially provided on the peripheral wall of the handle portion, the slideways are in parallel with a central axis of the trocar, and the control sliding blocks can be slidably disposed on the slideways.

6. The controlled multi-needle point expanding radiofrequency ablation electrode needle according to claim 5, characterized in that, the slideways are provided with a scale bar.

7. The controlled multi-needle point expanding radiofrequency ablation electrode needle according to claim 1, characterized in that, a plurality of sliding tracks are circumferentially provided in the handle portion, the metallic sliding rods are slidably provided on the sliding tracks.

8. The controlled multi-needle point expanding radiofrequency ablation electrode needle according to claim 7, characterized in that, the sliding tracks are provided with sliding beads, the metallic sliding rod is provided with a plurality of grooves at intervals in its sliding direction, and the sliding bead can be clipped within one of the plurality of grooves.

9. The controlled multi-needle point expanding radiofrequency ablation electrode needle according to claim 8, characterized in that, the grooves are five, a spacing between the grooves adjacent to each other is 1 cm, a diameter of an arc formed after the electrode sub-needles extend from the opening for extending of needles of the trocar and expand and bend outwardly is 1 cm to 5 cm.

10. The controlled multi-needle point expanding radiofrequency ablation electrode needle according to claim 1, characterized in that, needle tip of the electrode sub-needle is provided with a thermocouple temperature sensor.

11. The controlled multi-needle point expanding radiofrequency ablation electrode needle according to claim 2, characterized in that, a plane where the electrode sub-needle expanding from the opening for extending of needles expands and bends outwardly is co-planar with a sagittal plane of the control sliding block connected to the expanding electrode sub-needle.
